(19) United States
(12) Patent Application Publication
(10) Pub. No.: US 2022/0307038 A1
(43) Pub. Date: Sep. 29, 2022

(54) IMPROVED METHODS AND COMPOSITIONS FOR INCREASED DOUBLE STRANDED RNA PRODUCTION

(71) Applicant: RNAISSANCE AG LLC, Shawnee Mission, KS (US)

(72) Inventor: Anil Kumar, St. Louis, MO (US)

(73) Assignee: RNAISSANCE AG LLC, Shawnee Mission, KS (US)

(21) Appl. No.: 17/615,513

(22) PCT Filed: May 29, 2020
(86) PCT No.: PCT/US2020/035357

§ 371 Date: Nov. 30, 2021

Related U.S. Application Data
(60) Provisional application No. 62/854,843, filed on May 30, 2019.

(51) Int. Cl. C12N 15/67; C12N 15/70; C07K 14/005

(52) U.S. Cl. CPC C12N 15/67; C12N 15/70; C07K 14/005; C12N 2795/18122

(57) ABSTRACT

The invention provides methods and compositions for improved production of large quantities of unencapsidated doublestrand RNA (dsRNA) in vivo. The disclosed methods and compositions, comprising co-expression of genes encoding orotate phospori-bosyl transferase, bacteriophage coat protein and dsRNA produce a significant improvement over current in vivo methods of producing unencapsidated dsRNA.

### CROSS-REFERENCE TO RELATED APPLICATIONS

This application claims the benefit of U.S. Provisional Application No. 62/854,843, filed May 30, 2019, the entire contents of which are incorporated herein by reference.

### FIELD OF THE INVENTION

The present invention relates to improved methods and compositions for increasing in vivo production of unencapsidated double-stranded RNA.

### BACKGROUND OF THE INVENTION

The ability to suppress gene expression with RNA homologous to targeted gene sequences has greatly increased demand for large scale production of such RNA. However, the chemical fragility of RNA limits commercial development of many of these techniques. Large scale production of purified RNA is constrained by the high costs associated with in vitro synthesis methods and by the low yields and high processing requirements associated with in vivo methods.

The susceptibility of RNA to enzymatic and environmental degradation varies widely depending on the nature of the RNA molecule. Single-stranded RNA (ssRNA) is extremely sensitive to degradation and in vivo production of such molecules requires use of production strains lacking endogenous RNAses and benefits by coupling production of the RNA to encapsidation within viral capsid shells to produce Virus-Like Particles (VLPs). Encapsidation reduces degradation of RNA during production and allows more aggressive treatment during purification. VLPs effectively preserve such fragile RNA from degradation by sequestering the RNA within a relatively inert protein shell. Double stranded RNA (dsRNA) are somewhat less susceptible to degradation by cellular and environmental RNAses, although the highest in vivo yields of dsRNA also involve production strains lacking RNAses and many dsRNA also benefit from encapsidation. Unfortunately, the semi-rigid nature of the double-stranded stem region of dsRNA limits the range of dsRNA that can be encapsidated, since the length of the double-stranded stem structure cannot exceed the interior diameter of the capsid.

In the course of exploring techniques for increasing the range of dsRNA stems that may be encapsidated, the inventors discovered that under certain conditions a significant amount of unencapsidated dsRNA can be recovered directly from cell lysates, but only when the host cells co-express capsid protein. Such methods and compositions may be adapted for commercial scale production of dsRNA, and are disclosed in international patent publication WO 2017/160600, the contents of which are hereby incorporated by reference in its entirety.

While developing the initial technology for increased unencapsidated dsRNA production, the inventors made a surprising additional discovery; use of a host strain with increased orotate phosphoribosyltransferase activity, encoded by the pyrE gene, significantly increases the amount of unencapsidated dsRNA produced by the original method. Without being bound by theory, increasing orotate phosphoribosyltransferase activity by increasing expression of the pyrE gene product suggests a model wherein the increased capacity of the host cell to channel pyrimidine biosynthesis to uracil formation thereby improving the overall yield of dsRNA. However, the improvement in dsRNA yield under such conditions was found to be entirely dependent on overexpression of the pyrE gene product itself and not necessarily on increased availability of uracil, since addition of endogenous uracil (alone or in combination with other nucleotides) does not improve dsRNA yields in the absence of increased pyrE expression.

Regardless of the exact mechanism, the improvement of unencapsidated dsRNA production in the presence of bacteriophage coat protein in a microbial host cell overexpressing the pyrE gene product is quite large. The methods and compositions described here represent a significant improvement over the production of unencapsidated double stranded RNA in the presence of bacteriophage coat protein described in WO 2017/160600.

### SUMMARY OF THE INVENTION

The invention described in the following embodiments provides methods and compositions for improved production of large quantities of unencapsidated dsRNA in vivo. The disclosed methods and compositions represent a significant improvement over current in vivo methods of producing dsRNA.

### DESCRIPTION OF THE DRAWINGS

FIG. 1. Agarose gel of double-stranded RNA (dsRNA) produced from cells containing plasmids pAPSE10379, pAPSE10447 or pAPSE10448, as indicated. One hundred nanograms RNAse/Proteinase K treated RNA samples were run on 1.6% Agarose gel. Target dsRNA yields were estimated by comparing the intensity of the target RNA band with the standard containing known amounts of DNA using the quantitation tool provided in Bio-Rad Image Lab 4.01 software.

FIG. 2. Agarose gel of dsRNA produced from cells containing plasmids pAPSE10379, pAPSE10448, or pAPSE10458, as indicated. One hundred nanograms of RNAse/Proteinase K treated RNA samples were run on 1.6% Agarose gel. Target dsRNA yields were estimated by comparing the intensity of the target RNA band with the standard containing known amounts of DNA using the quantitation tool provided in Bio-Rad Image Lab 4.01 software.

FIG. 3. Agarose gel of dsRNA produced from cells containing plasmids pAPSE10448 or pAPSE10471, as indicated. One hundred nanograms of RNAse/Proteinase K treated RNA samples were run on 1.6% Agarose gel. Target dsRNA yields were estimated by comparing the intensity of the target RNA band with the standard containing known amounts of DNA using the quantitation tool provided in Bio-Rad Image Lab 4.01 software.

FIG. 4. Agarose gel illustrating the effect of addition of exogenous Uracil or all four ribonucleotides, as indicated, on the yield of target dsRNA from cells containing plasmid pAPSE10379. Plasmid pAPSE10448 was included as a control in the experiment. One hundred nanograms of RNAse/Proteinase K treated RNA samples were run on 1.6% Agarose gel. Target dsRNA yields were estimated by comparing the intensity of the target RNA band with the standard containing known amounts of DNA using the quantitation tool provided in Bio-Rad Image Lab 4.01 software.

### DETAILED DESCRIPTION OF THE INVENTION

The present invention comprises improved compositions and methods for producing large quantities of dsRNA in vivo from microbial cells. In its most basic form, the invention involves co-expressing pyrE and a bacteriophage capsid protein in conjunction with the desired dsRNA for a period of time sufficient to allow accumulation of the dsRNA in a host cell, lysing the host cell and then recovering intact unencapsidated dsRNA directly from the cell lysate. Microbial host cells expressing only the endogenous level of the pyrE gene product produce significantly lower levels of unencapsidated dsRNA in the presence of bacteriophage coat protein.

A number of permutations for expressing the pyrE, the coat protein and the dsRNA are contemplated under the current invention. In one permutation all three genes are expressed from a single inducible transcriptional promoter. In another permutation the pyrE gene and the coat protein gene are expressed from an inducible promoter separate from the promoter transcribing the dsRNA sequence. In this instance the promoter transcribing the pyrE gene and the coat protein may be induced prior to induction of the dsRNA promoter transcribing the dsRNA, to allow expression of orotate phosphoribosyltransferase and coat protein to accumulate within the host cell prior to dsRNA accumulation. In still other permutations, the coat protein and pyrE gene may be transcribed from separate transcriptional promoters and may be induced at different times to allow differential accumulation of the respective gene products. The coat protein and pyrE coding sequences may be placed downstream of different ribosome binding site sequences to differentially modulate protein synthesis.

Growth of cells containing the dsRNA, coat protein and recombinant pyrE gene may be carried out in a minimal (mineral) media or in a rich media. Such media are well known to those of ordinary skill in the art. The invention as disclosed herein may be carried out using standard industrial microbiology techniques and standard fermentation procedures, so long as such methods are adapted to the specific plasmid and host cell requirements, such as providing the appropriate selection markers to retain the specific plasmid vectors, using the appropriate stimuli to induce transcription of the specific promoters at appropriate times, and maintain the required temperature and respiratory conditions necessary for cell growth, each of which is within the working knowledge of those of ordinary skill in the art.

### A. DEFINITIONS

As used herein, the terms “capsid protein” or “coat protein” refers to the coat protein of bacteriophage MS2 or bacteriophage Q13, capable of binding the cognate bacteriophage RNA pac site with high affinity and assembling into a complex hollow tertiary structure in which the bacteriophage RNA may be entirely encapsidated within the hollow tertiary structure. The term “capsid” refers to the hollow tertiary structure formed by assembly of individual capsid proteins. An incomplete capsid is understood to mean a capsid that is not completely closed, such that no hollow tertiary structure is formed.

As used herein “ssRNA” and “dsRNA” refer to “single-stranded RNA and double stranded RNA, respectively. A ssRNA is comprised of an RNA sequence of any length that lacks sufficient internal homology to form any significant secondary structures such as hairpins or other structures dependent on hybridization of internal complementary sequences with one another via Watson-Crick base pairing of nucleotide bases within the complementary sequences. In contrast, a dsRNA comprises RNA sequences with sufficient internal homology to form significant secondary structures such as hairpins due to hybridization of internal complementary sequences with one another via Watson-Crick base pairing of nucleotide bases within the complementary sequences.

As used herein “unencapsdiated dsRNA” means double strand RNA not incorporated within capsids and includes both dsRNA associated with incomplete capsids and dsRNA with no association with bacteriophage coat protein whatsoever. The dsRNA contemplated in the present invention comprises a single RNA with two complementary domains separated by a nonhomologous recombinant spacer/loop sequence capable of forming a hairpin structure.

### B. COMMON MATERIALS, AND METHODS

Routine microbial and molecular cloning methods and tools, including those for generating and purifying DNA, RNA, and proteins, and for transforming host organisms and expressing recombinant proteins and nucleic acids as described herein, are fully within the capabilities of a person of ordinary skill in the art and are well described in the literature. See, e.g., Sambrook, et al., Molecular Cloning: A Laboratory Manual, 2nd ed., Cold Spring Harbor Laboratory Press, Cold Spring Harbor, N.Y. (1989); Davis, et al., Basic Methods in Molecular Biology, Elsevier Science Publishing Co., Inc., N.Y. (1986); and Ausubel, et al., Current Protocols in Molecular Biology, Greene Publ. Assoc., Wiley-Interscience, N.Y. (1995). The disclosures in each of which are herein incorporated by reference.

Each of the recombinant DNA constructs described in further detail below are based on a common plasmid vector series derived from plasmid pBR322. Transformation of the plasmids described herein into host cells capable of inducible expression of T7 polymerase produces cell lines capable of expressing RNA transcripts. All such strains are referred to generally herein as “expression strains”. Unless otherwise indicated, each of the plasmids described herein was electroporated into E. coli strain HT115(DE3) with genotype F⁻, mcrA, mcrB, IN (rrnD-rrnE)1,rnc14::Tn10 (Lambda DE3 lysogen: lacUV5 promoter-T7 polymerase)) and the resulting recombinant transformants were selected on LB agar plates containing 12 μg/ml tetracycline and 100 μg/ml ampicillin. Single colonies were isolated, the presence of intact plasmid confirmed by restriction enzyme analysis and the confirmed transformed cells archived for future use.

Standard expression studies comprised inoculating transformed cells into 100 ml of LB broth containing 100 μg/ml ampicillin and incubating the cultures with vigorous shaking at 37° C. When the culture density reached OD₆₀₀ of 0.8, inducer (isopropyl-β-D-thiogalactopyranoside (Gold Biotech, St. Louis, MO)) was added to a final concentration of 1 mM. Cells were harvested four hours post-induction by centrifugation at 3,000 g at 4° C. for 30 minutes and stored on ice until lysis.

RNA was isolated from harvested cells by resuspending a 5 ml equivalent of cell culture of harvested cells in sonication buffer comprising Tris-HCl pH 7, 10 mM NaCl and sonicating the suspended cells on ice for 3 minutes. Cell debris was removed by centrifugation at 16,000 g the supernatant (cleared lysate) was immediately processed to recover RNA and VLPs as described. RNA was recovered from half of the cleared lysate using the commercial Purelink RNA Mini Kit method (Ambion Cat. No. 12183018A, Thermo Fisher Scientific Inc., Waltham, Mass.) according to the manufacturer's instructions.

RNA from isolated in this manner was dissolved in 50 μl of nuclease-free water. To determine the concentration of dsRNA in a sample, the samples were treated with RNAse A (Invitrogen Cat. No. AM2274, Thermo Fisher Scientific Inc.) to degrade single stranded RNA under the manufacturers recommended conditions, the concentration of dsRNA was determined spectrophotometrically. One hundred nanograms of each RNA sample was loaded onto 1.6% Agarose gels and one lane of each gel was loaded with dsDNA size markers of known concentration and the samples were electrophoresed. The gels were stained with ethidium bromide and each band quantitated by densitometry using the dsDNA markers as a standard curve as described in the Figure legends.

Little or no differences in final cell densities were observed between the cultures from which the samples were harvested and in all cases the cultures appear to have reached stationary phase prior to harvest.

### C. PREFERRED EMBODIMENTS

The following are among the preferred embodiments of the invention.

In one embodiment the invention comprises a microbial cell overexpressing the pyrE gene encoding orotate phosphoribosyltransferase, a bacteriophage coat protein gene encoding a capsid protein, and a gene encoding a dsRNA comprising a self-complementary stretch of sequence separated by non-complementary sequence such that upon hybridization of the complementary sequences a stem-loop structure is formed, wherein the stem portion of the molecule functions as an RNAi precursor and further comprising a bacteriophage pac site signal. Expression of the pyrE gene product, the bacteriophage coat protein gene, and the dsRNA gene results in increased accumulation of un-degraded unencapsidated dsRNA. The amount of dsRNA produced in this way greatly exceeds the amount of dsRNA produced in a microbial cell lacking high level expression of the pyrE gene product but including high level expression of the bacteriophage coat protein and the dsRNA gene.

In one embodiment the bacteriophage capsid protein expressed in conjunction with the pyrE gene is encoded by the coat protein gene of a species of leviviridae. In a preferred embodiment the coat protein gene encodes the capsid protein of bacteriophage MS2. In another preferred embodiment the coat protein gene encodes the capsid protein of bacteriophage Qbeta.

In an embodiment the capsid protein expressed in conjunction with the pyrE gene comprises the N-terminus of the leviviridae capsid protein. In another embodiment the capsid protein comprises the N-terminal 41, 35, 25, 21 or 12 amino acids of the MS2 capsid protein. In another embodiment the capsid protein comprises the N-terminal 41, 35, 25, 21 or 12 amino acids of the Qbeta capsid protein.

In an embodiment the gene encoding the dsRNA may be associated with and expressed from an inducible transcriptional promoter. The coat protein gene and the pyrE gene may be associated with and expressed from a constitutive or inducible transcriptional promoter, together as a single transcript or individually from different transcriptional promoters. The inducible transcriptional promoter associated with expression of the dsRNA may be the same inducible transcriptional promoter or a different transcriptional promoter from a transcriptional promoter associated with expression of the coat protein gene and/or the pyrE gene. In one embodiment the inducible transcriptional promoter or promoters associated with expression of the coat protein gene and the pyrE gene is induced before induction of the inducible transcriptional promoter associated with expression of the dsRNA to allow accumulation of capsid protein and orotate phosphoribosyltransferase prior to production of dsRNA. In another embodiment the transcriptional promoter or promoters associated with expression of the coat protein gene and the pyrE gene comprise constitutive transcriptional promoter or promoters.

In an embodiment the gene encoding the dsRNA, the pyrE gene, and the coat protein gene encoding the capsid protein are present on a plasmid or extrachromosomal element. The gene encoding the dsRNA, the pyrE gene and the coat protein gene may all be present on the same plasmid or extrachromosomal element or may be present on separate plasmids or extrachromosomal elements. In other embodiments the gene encoding the dsRNA and the pyrE gene encoding orotate phosphoribosyltransferase are present on a plasmid or extrachromosomal element. The gene encoding the dsRNA and the pyrE gene may be present on the same plasmid or extrachromosomal element or may be present on separate plasmids or extrachromosomal elements. In still other embodiments the gene encoding the pyrE gene encoding orotate phosphoribosyltransferase and the coat protein gene encoding the capsid protein are present on a plasmid or extrachromosomal element. The gene encoding the pyrE gene and the coat protein gene may be present on the same plasmid or extrachromosomal element or may be present on separate plasmids or extrachromosomal elements. In yet other embodiments one or more of the genes encoding the dsRNA, the coat protein, and pyrE gene may be present on the microbial host cell chromosome or chromosomes.

In related embodiments, the dsRNA may be purified from the microbial host cell overexpressing the pyrE and coat protein gene products by lysing the cells to produce a lysate and purifying the dsRNA from the cellular constituents within the lysate prior to processing the purified dsRNA for application. Such processing may include, but is not limited to, mixing with excipients, binders or fillers to improve physical handling characteristics, stabilizers to reduce degradation, or other active agents such as chemical pesticides, fungicides, defoliants or other RNAi molecules to broaden the spectrum of application targets, and may include pelletizing, spray drying or dissolving the materials into liquid carriers. In another embodiment the dsRNA is not further purified from the lysate but is processed directly for application. In still another embodiment the microbial host cell is not lysed but is processed directly for application and the dsRNA remains unpurified within the processed cells.

### EXAMPLES

The following Examples are meant to be illustrative and are not intended to limit the scope of the invention as set forth in the appended Claims.

### Example 1

Increased expression of pvrE in conjunction with bacteriophage MS2 coat protein increases dsRNA production.

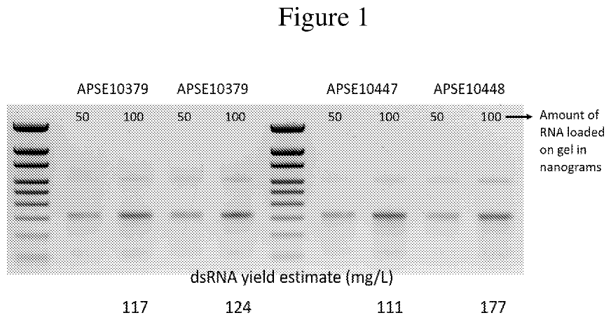

To test expression of pyrE on dsRNA yields, the production plasmid, pAPSE10379 (SEQ ID NO: 1) was modified by cloning the T1-T2 terminator as a SalI-Nrul fragment into the corresponding sites while adding an AvrII site downstream of the SalI site but upstream of the rrnB terminator to create pAPSE10424 (SEQ ID NO: 2). The pyrE gene coding sequence coupled to a strong E. coli ribosome binding site (sequence AGAAGGA) was than cloned as a SalI-AvrII fragment into the corresponding sites downstream of the T7 promoter expressing the MS2 coat protein gene in pAPSE10424 to create pAPSE10448 (SEQ ID NO: 3). In this plasmid the MS2 coat protein gene and the pyrE gene are transcribed as a single transcript from the strong inducible T7 promoter. About 50 to 100% increase in dsRNA yield was observed in cells containing construct pAPSE10448 relative to those containing pAPSE10379 upon induction (FIG. 1, Table 1, and Table 2)

### Example 2

Effect on dsRNA production of expression of pvrE under the control of a pyrimidine regulated promoter.

Expression of the pyrC gene encoding the pyrimidine biosynthetic enzyme dihydroorotase is regulated by transcription start site switching and translation control. The primary regulatory effector of pyrC expression is cytidine nucleotide (CTP). Nucleotide-sensitive selection of transcription start sites is used to produce alternative transcripts with different potentials for translation. When the intracellular level of CTP is high, transcripts with hidden ribosome binding sites are produced. In contrast, when the CTP levels are low and GTP levels high, transcripts that are readily translated are produced. Hence, the pyrC gene is transcribed and translated when pyrimidine levels are low.

When placed under the control of the pyrC promoter, pyrE expression is expected to be turned on upon demand for pyrimidines. A recombinant fragment comprising the pyrE gene coding sequence under the control of the pyrC promoter was cloned as a Sal1-AvrII fragment into the corresponding sites, downstream of the T7 expression cassette driving MS2 coat protein gene in pAPSE10424 to create plasmid pAPSE10447 (SEQ ID NO: 4). In this plasmid MS2 coat protein expression is under the control of a T7 promoter while that of the pyrE gene is under the control of the pyrC promoter.

TABLE 1
Target dsRNA yields of non-pyrE (pAPSE10379) and pyrE
containing constructs(pAPSE10447 and pAPSE10448) in shake flask
experiments with minimal media (based on yields from FIG. 1).

| | Spacer | RNA | Encapsid | Excapsid |
|---|---|---|---|---|
| Plasmid No. | Size (bp) | size (bp) | yield (mg/L) | yield (mg/L) |
| APSE10379 | 166 | 300 | <2 | ~120 |
| APSE10447 | 166 | 300 | <2 | ~111 |
| APSE10448 | 166 | 300 | <2 | ~177 |

The results presented in Table 1 are derived from shake flask studies of cells grown in minimal media and are thus the result of cultures with relatively low cell concentration (approximately 1-2 OD₆₀₀) corresponding to approximately 10⁹ to 10¹⁰ cells/ml. Much higher cell densities (20 to 30 fold higher, corresponding to 2-3×10¹⁰ cells/ml) can easily be achieved in regulated fermentations using modern bioreactor techniques. Higher cell densities translate into increased overall volumetric yields. Conservative estimates of total volumetric yields of dsRNA from the systems described herein at high cell densities are presented in Table 2.

TABLE 2
Predicted dsRNA yields of non-pyrE (APSE10379) and pyrE construct
(APSE10448) in fermenter/bioreactor experiments with minimal media.

| | Spacer | RNA | Encapsid | Excapsid |
|---|---|---|---|---|
| Plasmid No. | Size (bp) | size (bp) | yield (mg/L) | yield (mg/L) |
| APSE10379 | 166 | 300 | <50 | 2000 to 2700 |
| APSE10448 | 166 | 300 | <50 | 4000 to 4800 |

### Example 3

Effect on dsRNA production of expression of pvrE under the control of a dedicated inducible promoter.

To further explore the utility of inducible promoters driving expression of pyrE to improve dsRNA a SalI-AvrII fragment containing the pyrE gene coding sequence under the control of T7 promoter with a strong E.coli ribosome binding site was cloned into the corresponding sites of plasmid pAPSE10424 to create pAPSE10471 (SEQ ID NO: 5). In this plasmid expression of the MS2 coat protein gene and the pyrE gene are driven by separate T7 promoters. As shown in Table 3, RNA yields similar to those observed with pAPSE10448 (in which the MS2 coat protein and the pyrE gene are transcribed from the same T7 promoter).

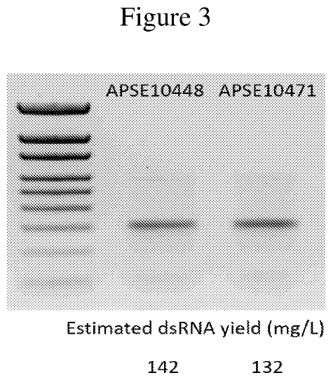

TABLE 3
Target dsRNA yields of pyrE constructs with pyrE gene independently
driven by T7 promoter (APSE10471) vs pyrE transcribed as
read-through transcription downstream of MS2 coat protein (APSE10448)
in minimal media (based on yields from FIG. 3).

| | Spacer | RNA | Encapsid | Excapsid |
|---|---|---|---|---|
| Plasmid No. | Size (bp) | size (bp) | yield (mg/L) | yield (mg/L) |
| APSE10448 | 166 | 300 | <2 | ~140 |
| APSE10471 | 166 | 300 | <2 | ~135 |

### Example 4

Effect on dsRNA production of expression of pvrE of varvin2 ribosome binding sites.

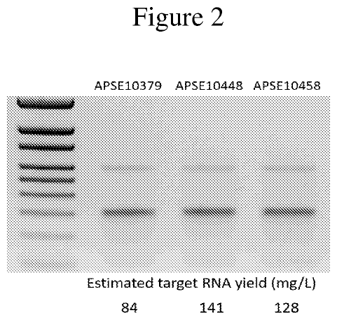

To examine how modulating translation of the pyrE gene might affect dsRNA production pAPSE10424 was modified by ligation of a SalI-AvrII fragment comprising the pyrE gene coding sequence with a weak E.coli ribosome binding site (sequence AGGA) downstream of the T7 expression cassette driving MS2 coat protein gene to create pAPSE10458 (SEQ ID NO: 6). In this plasmid the MS2 coat protein and the pyrE gene are transcribed as a single transcript from the strong T7 promoter upon induction, but the expression of the pyrE gene is reduced relative to pAPSE10448 due to the weaker ribosome binding site present in pAPSE10458. Cells expressing pAPSE10458 exhibit a 53% increase in dsRNA yield relative to pAPSE10379 (FIG. 2, Table 4).

TABLE 4
Target dsRNA yields of non-pyrE (APSE10379) and pyrE constructs
with strong (APSE10448) vs weak (APSE10458) ribosome
binding sites in minimal media (based on yields from FIG. 2).

| | Spacer | RNA | Encapsid | Excapsid |
|---|---|---|---|---|
| Plasmid No. | Size (bp) | size (bp) | yield (mg/L) | yield (mg/L) |
| APSE10379 | 166 | 300 | <2 | ~84 |
| APSE10448 | 166 | 300 | <2 | ~140 |
| APSE10458 | 166 | 300 | <2 | ~128 |

### Example 5

Effect of exogenous uracil and other nucleotides.

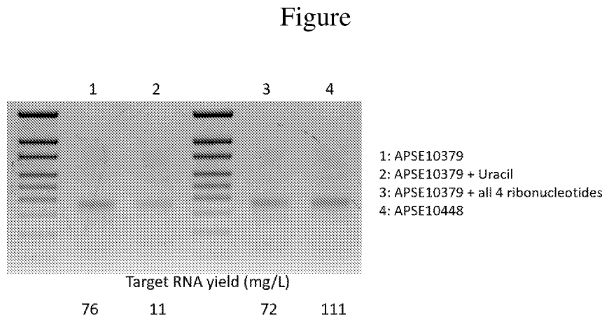

Plasmids pAPSE10379 and pAPSE10448 were used in this experiment. Plasmid pAPSE10448 expresses both the MS2 coat protein and the pyrE gene as a single transcript by the strong inducible T7 promoter, whereas pAPSE10379 lacks the pyrE gene, but is otherwise identical to pAPSE10448. To investigate whether the increase in dsRNA yield observed when endogenous orotate phosphoribosyltransferase activity is induced (as in cells containing pAPSE10448) can be replaced merely by increasing intracellular levels of uracil, the minimal culture media of cells containing pAPSE10379 was supplemented with uracil alone or all four nucleotides and the amount of dsRNA produced upon induction of coat protein and the dsRNA itself was measured. A culture flask of cells expressing pAPSE10379 was grown and induced in minimal growth medium without any supplementation as a control culture. A second flask, containing the same cells in minimal medium supplemented with 1 gram/L of uracil, and a third flask containing the same cells supplemented with all 1 gram/L of each of the four nucleotides (adenosine, uracil, guanosine, and cytosine) were added to the third flask at 1 gram/L level for each of the four ribonucleotides. A culture of cells containing pAPSE10448 culture was grown in minimal medium lacking any supplementation. As shown in FIG. 4 and in Table 5 below, increasing endogenous orotate phosphoribosyltransferase in conjunction with bacteriophage coat protein produces only about 32% more dsRNA than that produced by cells supplemented with all four ribonucleotides. Surprisingly, supplementation with uracil alone reduces the amount of dsRNA produced by the cells to approximately 10% of the level observed when orotate phosphoribosyltransferase activity is increased. Indeed, uracil appears to suppress dsRNA synthesis to only about 15% of the level observed in the presence of all four nucleotides or in the absence of any supplementation at all. Thus, improved production of dsRNA requires increased expression of orotate phosphoribosyltransferase in conjunction with bacteriophage coat protein per se, the improvement in yield not being merely a function of increased ribonucleotide availability.

TABLE 5
Target dsRNA yields of non-pyrE
construct APSE10379 with addition of
exogenous Uracil/all four
ribonucleotides (based on yields from FIG. 4).

| | Spacer | RNA | Addition of | Excapsid |
|---|---|---|---|---|
| | Size | size | Uracil or 4 | yield |
| Plasmid No. | (bp) | (bp) | nucleotides (1 g/L) | (mg/L) |
| APSE10379 | 166 | 300 | No | ~76 |
| APSE10379 | 166 | 300 | Uracil | ~11 |
| APSE10379 | 166 | 300 | All 4 nucleotides | ~72 |
| APSE10448 | 166 | 300 | No | ~111 |

1-5.(canceled)

6. A modified bacterial cell for producing dsRNA in vivo, the modified bacterial cell comprising:
a. a genetic modification for increasing expression of a pyrE gene;
b. a nucleic acid construct comprising a nucleic acid sequence encoding a double-stranded RNA (dsRNA) operably linked to an expression control sequence; and
c. a nucleic acid construct comprising a nucleic acid sequence encoding a capsid protein operably linked to an expression control sequence.

7. The modified bacterial cell of claim 6, wherein the dsRNA is selected from the group consisting of siRNA, shRNA, sshRNA, and miRNA.

8. The modified bacterial cell of claim 6, wherein the capsid protein is a leviviridae coat protein gene encoding a capsid protein.

9. The modified bacterial cell of claim 6, wherein the capsid protein is a capsid protein of bacteriophage MS2 or N-terminal 41, 35, 25, 21 or 12 amino acids of the MS2 capsid protein.

10. The modified bacterial cell of claim 6, wherein the capsid protein is a capsid protein of bacteriophage Qβ or N-terminal 41, 35, 25, 21 or 12 amino acids of the Qβ capsid protein.

11. The modified bacterial cell of claim 6, wherein the bacterial cell is an E. coli K-12 strain comprising a frameshift mutation in a rph gene.

12. The modified bacterial cell of claim 11, wherein the E. coli K-12 strain comprising a frameshift mutation in the rph gene comprises orotate phosphoribosyltransferase (ORPTase) with a specific activity of about 5-20 units and wherein the E. coli K-12 strain comprising a frameshift mutation in the rph gene and the genetic modification for increasing the expression of the pyrE gene comprises ORPTase with a specific activity of at least about 30 units or about 30-90 units.

13. The modified bacterial cell of claim 11, wherein the genetic modification for increasing the expression of the pyrE gene comprises a correction of the frameshift mutation in the rph gene.

14. The modified bacterial cell of claim 11, wherein the genetic modification for increasing the expression of the pyrE gene comprises a deletion of the rph gene.

15. The modified bacterial cell of claim 11, wherein the genetic modification for increasing the expression of the pyrE gene comprises a replacement of the rph gene comprising the frameshift mutation with a nucleic acid sequence encoding an rph gene from an E. coli strain that does not comprise the frameshift mutation.

16. The modified bacterial cell of claim 6, wherein the genetic modification for increasing the expression of the pyrE gene comprises an exogenous nucleic acid construct encoding the pyrE gene operably linked to a promoter.

17. The modified bacterial cell of claim 6, further comprising dsRNA encoded by the nucleic acid sequence encoding the dsRNA, wherein levels of the dsRNA are increased when compared to the levels of dsRNA in a bacterial cell before the expression of the pyrE gene is increased.

18. The modified bacterial cell of claim 6, wherein the modified bacterial cell is a modified E. coli K-12 strain MG1655 (ATCC No. 47076), strain HD115 (DE3), or W3110 strain (ATTC No. 27325).

19. The modified bacterial cell of claim 6, wherein the genetic modification for increasing the expression of the pyrE gene comprises a nucleic acid construct comprising an exogenous nucleic acid sequence encoding the pyrE gene operably linked to a promoter, and wherein the nucleic acid construct comprising the exogenous nucleic acid sequence encoding the pyrE gene operably linked to a promoter, the nucleic acid construct comprising a nucleic acid sequence encoding a dsRNA operably linked to an expression control sequence, and the nucleic acid construct comprising a nucleic acid sequence encoding a capsid protein operably linked to an expression control sequence are comprised on plasmid pAPSE10448 (SEQ ID NO: 3), plasmid pAPSE10447 (SEQ ID NO: 4), or plasmid pAPSE10471 (SEQ ID NO: 5).

20. A method for producing dsRNA in vivo, the method comprising expressing the dsRNA with a gene encoding a bacteriophage capsid protein in a modified bacterial cell comprising a genetic modification for increasing the expression of a pyrE gene.

21. The method of claim 20, wherein the dsRNA is selected from the group consisting of siRNA, shRNA, sshRNA, and miRNA.

22. The method of claim 20, wherein the capsid protein is a capsid protein of bacteriophage MS2, N-terminal 41, 35, 25, 21 or 12 amino acids of the MS2 capsid protein, capsid protein of bacteriophage Qβ or N-terminal 41, 35, 25, 21 or 12 amino acids of the Qβ capsid protein.

23. The method of claim 20, wherein the bacterial cell is an E. coli K-12 strain comprising a frameshift mutation in a rph gene.

24. The method of claim 20, wherein expressing the dsRNA with a gene encoding bacteriophage capsid protein comprises expressing the dsRNA from a nucleic acid construct comprising a nucleic acid sequence encoding the dsRNA operably linked to an expression control sequence and expressing the capsid protein from a nucleic acid construct comprising a nucleic acid sequence encoding the capsid protein operably linked to an expression control sequence.

25. The method of claim 20, further comprising modifying the expression of the pyrE gene by introducing into the bacterial cell a nucleic acid construct comprising an exogenous nucleic acid sequence encoding the pyrE gene operably linked to a promoter.

26. The method of claim 25, wherein modifying the expression of the pyrE gene and expressing the dsRNA and the capsid protein comprises introducing plasmid pAPSE10448 (SEQ ID NO: 3), plasmid pAPSE10447 (SEQ ID NO: 4), or pAPSE10471 (SEQ ID NO: 5) into the bacterial cell, wherein the plasmid comprises the nucleic acid construct comprising the exogenous nucleic acid sequence encoding the pyrE gene operably linked to a promoter, a nucleic acid construct comprising a nucleic acid sequence encoding the dsRNA operably linked to an expression control sequence, and a nucleic acid construct comprising a nucleic acid sequence encoding the capsid protein operably linked to an expression control sequence.

27. The method of claim 20, further comprising purifying the dsRNA from the bacterial cells by lysing the cells to produce a lysate and purifying the dsRNA from cellular constituents within the lysate prior to processing the purified dsRNA for application.

28. The method of claim 20, further comprising lysing the bacterial cell to produce a lysate, wherein the dsRNA is not further purified from the lysate prior to processing for application.


SEQUENCE LISTING (7 sequences)

SEQ ID NO: 1 - DNA, 5688 bp, Artificial Sequence
  Recombinant plasmid pAPSE10379
  promoter (10)..(28): Bacteriophage T7 gene 1 promoter
  protein_bind (32)..(50): Bacteriophage MS2 coat protein binding site
  misc_feature (59)..(358): Solenopsis invicta actin sense strand gene fragment
  misc_feature (367)..(516): recombinant spacer/loop sequence
  misc_feature (525)..(823): Solenopsis invicta actin antisense strand gene fragment
  protein_bind (899)..(917): Bacteriophage MS2 coat protein binding site
  terminator (933)..(980): Bacteriophage T7 terminator
  promoter (1306)..(1324): Bacteriophage T7 gene 1 promoter
  misc_feature (1397)..(1786): Bacteriophage MS2 coat protein gene
  terminator (1819)..(1866): Bacteriophage T7 terminator
  misc_feature (1978)..(1978): Sal I restriction site
  misc_feature (2301)..(2301): Nru I restriction site
  misc_feature (4620)..(5585): beta-lactamase gene
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 2 - DNA, 5751 bp, Artificial Sequence
  Recombinant plasmid pAPSE10424
  promoter (10)..(28): Bacteriophage T7 gene 1 promoter
  protein_bind (32)..(50): Bacteriophage MS2 coat protein binding site
  misc_feature (59)..(358): Solenopsis invicta actin sense strand gene fragment
  misc_feature (367)..(516): recombinant spacer/loop sequence
  misc_feature (525)..(823): Solenopsis invicta actin antisense strand gene fragment
  protein_bind (899)..(917): Bacteriophage MS2 coat protein binding site
  terminator (933)..(980): Bacteriophage T7 terminator
  promoter (1306)..(1324): Bacteriophage T7 gene 1 promoter
  misc_feature (1397)..(1786): Bacteriophage MS2 coat protein gene
  terminator (1819)..(1866): Bacteriophage T7 terminator
  misc_feature (1978)..(1978): Sal I restriction site
  misc_feature (1986)..(1986): Avr II restriction site
  terminator (2145)..(2350): E. coli rrnB terminator
  misc_feature (2364)..(2364): Nru I restriction site
  misc_feature (4683)..(5648): beta-lactamase gene
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgacatcctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 3 - DNA, 6407 bp, Artificial Sequence
  Recombinant plasmid pAPSE10448
  promoter (10)..(28): Bacteriophage T7 gene 1 promoter
  protein_bind (32)..(50): Bacteriophage MS2 coat protein binding site
  misc_feature (60)..(358): Solenopsis invicta actin sense strand gene fragment
  misc_feature (367)..(516): recombinant spacer/loop sequence
  misc_feature (525)..(824): Solenopsis invicta actin antisense strand gene fragment
  protein_bind (899)..(917): Bacteriophage MS2 coat protein binding site
  terminator (933)..(980): Bacteriophage T7 terminator
  promoter (1306)..(1324): Bacteriophage T7 gene 1 promoter
  misc_feature (1397)..(1786): Bacteriophage MS2 coat protein gene
  terminator (1819)..(1866): Bacteriophage T7 terminator
  misc_feature (1978)..(1978): Sal I restriction site
  misc_feature (2000)..(2641): E. coli pyrE gene
  misc_feature (2642)..(2642): Avr II restrictin site
  terminator (2801)..(3006): E. coli rrnB terminator
  misc_feature (3020)..(3020): Nru I restriction site
  misc_feature (5339)..(6304): beta-lactamase gene
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgacagaaggagatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 4 - DNA, 6478 bp, Artificial Sequence
  Recombinant plasmid pAPSE10447
  promoter (10)..(28): Bacteriophage T7 gene 1 promoter
  protein_bind (32)..(50): Bacteriophage MS2 coat protein binding site
  misc_feature (60)..(358): Solenopsis invicta actin sense strand gene fragment
  misc_feature (367)..(516): recombinant spacer/loop sequence
  misc_feature (525)..(824): Solenopsis invicta actin antisense strand gene fragment
  protein_bind (899)..(917): Bacteriophage MS2 coat protein binding site
  terminator (933)..(980): Bacteriophage T7 terminator
  promoter (1306)..(1324): Bacteriophage T7 gene 1 promoter
  misc_feature (1397)..(1786): Bacteriophage MS2 coat protein gene
  terminator (1819)..(1866): Bacteriophage T7 terminator
  misc_feature (1978)..(1978): Sal I restriction site
  promoter (1984)..(2070): E. coli pyrC promoter
  misc_feature (2071)..(2712): E. coli pyrE gene
  misc_feature (2713)..(2713): Avr II restriction site
  misc_feature (2872)..(3077): E. coli rrnB terminator
  misc_feature (3091)..(3091): Nru I restriction site
  misc_feature (5410)..(6375): beta-lactamase gene
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgacgccctttatttttcgtgcaaaggaaaacgtttccgcttatcctttgtgtccggcaaaaacatcccttcagccggagcatagagattaatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 5 - DNA, 6434 bp, Artificial Sequence
  Recombinant plasmid pAPSE10471
  promoter (10)..(28): Bacteriophage T7 gene 1 promoter
  protein_bind (32)..(50): Bacteriophage MS2 coat protein binding site
  misc_feature (60)..(358): Solenopsis invicta actin sense strand gene fragment
  misc_feature (367)..(516): recombinant spacer/loop sequence
  misc_feature (525)..(824): Solenopsis invicta actin antisense strand gene fragment
  protein_bind (899)..(917): Bacteriophage MS2 coat protein binding site
  terminator (933)..(980): Bacteriophage T7 terminator
  promoter (1306)..(1324): Bacteriophage T7 gene 1 promoter
  misc_feature (1397)..(1786): Bacteriophage MS2 coat protein gene
  terminator (1819)..(1866): Bacteriophage T7 terminator
  misc_feature (1978)..(1978): Sal I restriction site
  promoter (1984)..(2002): Bacteriophage T7 gene 1 promoter
  misc_feature (2027)..(2668): E. coli pyrE gene
  misc_feature (2669)..(2669): Avr II restriction site
  terminator (2828)..(3033): E. coli rrnB terminator
  misc_feature (3047)..(3047): Nru I restriction site
  misc_feature (5366)..(6331): beta-lactamase gene
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgactaatacgactcactatagggagaccacagaaggagatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 6 - DNA, 6406 bp, Artificial Sequence
  Recombinant plasmid pAPSE10458
  promoter (10)..(28): Bacteriophage T7 gene 1 promoter
  misc_feature (32)..(50): Bacteriophage MS2 coat protein binding site
  misc_feature (60)..(358): Solenopsis invicta actin sense strand gene fragment
  misc_feature (367)..(516): recombinant spacer/loop sequence
  misc_feature (525)..(824): Solenopsis invicta actin antisense strand gene fragment
  protein_bind (899)..(917): Bacteriophage MS2 coat protein binding site
  terminator (933)..(980): Bacteriophage T7 terminator
  promoter (1306)..(1324): Bacteriophage T7 gene 1 promoter
  misc_feature (1397)..(1786): Bacteriophage MS2 coat protein gene
  terminator (1819)..(1866): Bacteriophage T7 terminator
  misc_feature (1978)..(1978): Sal I restriction site
  misc_feature (1999)..(2640): E. coli pyrE gene
  misc_feature (2641)..(2641): Avr II restrictin site
  terminator (2800)..(3005): E. coli rrnB terminator
  misc_feature (3019)..(3019): Nru I restriction site
  misc_feature (5338)..(6303): beta-lactamase gene
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgacaaaaaggatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 7 - DNA, 7 bp, Artificial Sequence
  Synthetic
agaagga